(19) United States
(12) Patent Application Publication
(10) Pub. No.: US 2017/0350838 A1
(43) Pub. Date: Dec. 7, 2017

(54) ELECTRIC MEASURING CARTRIDGE, BIOLOGICAL SAMPLE ELECTRIC MEASURING DEVICE, AND BIOLOGICAL SAMPLE ELECTRIC MEASURING METHOD

(71) Applicant: SONY CORPORATION, TOKYO (JP)

(72) Inventors: YOICHI KATSUMOTO, TOKYO (JP); DAISUKE TERAKADO, SAITAMA (JP)

(21) Appl. No.: 15/540,339

(22) PCT Filed: Nov. 5, 2015
(86) PCT No.: PCT/JP2015/081103

§ 371 Date: Jun. 28, 2017

(30) Foreign Application Priority Data
Jan. 16, 2015 (JP) 2015-006546

(51) Int. Cl. G01N 27/07; G01N 33/86

(52) U.S. Cl. CPC G01N 27/07; G01N 33/86

(57) ABSTRACT

To provide an electric measuring cartridge capable of decreasing a risk of contact with a connecting unit. Provided is an electric measuring cartridge at least provided with a biological sample holding unit which accommodates a biological sample, at least a pair of electrodes fixed to the biological sample holding unit, a connecting unit which electrically connects the electrodes to an external circuit, and a protecting unit which protects the connecting unit.

### TECHNICAL FIELD

The present technology relates to an electric measuring cartridge for measuring an electric characteristic of a biological sample. In more detail, this relates to the electric measuring cartridge capable of decreasing a risk of contact with a connecting unit, a biological sample electric measuring device using the electric measuring cartridge, and a biological sample electric measuring method.

### BACKGROUND ART

An electric characteristic of a biological sample is measured, and a physical property of the biological sample is determined from a measured result and a type of a cell and the like included in the biological sample is determined (for example, refer to Patent Document 1). The electric characteristic to be measured includes a complex dielectric constant, frequency distribution (dielectric spectrum) thereof and the like. The complex dielectric constant and the frequency distribution thereof are calculated by measuring complex capacitance or complex impedance between electrodes by using a solution holder and the like provided with the electrode for applying voltage to solution, in general.

Also, for example, Patent Document 2 discloses technology of obtaining information regarding blood coagulation from the dielectric constant of blood; this discloses “a blood coagulation system analyzing device including: a pair of electrodes, applying means which applies alternating voltage to the pair of electrodes at a predetermined time interval, measuring means which measures a dielectric constant of blood arranged between the pair of electrodes, and analyzing means which analyzes a degree of action of a blood coagulation system by using the dielectric constant of the blood measured at the time interval after an anticoagulant effect acting on the blood is ended”.

As an electric measuring container of the biological sample, Patent Document 3 discloses “an electric measuring container of a biological sample in a liquid state at least including a biological sample holding unit formed of resin for accommodating the biological sample in a liquid state, and an electric conducting unit fixed to the biological sample holding unit, in which the biological sample holding unit and the electric conducting unit are integrally formed in a state in which a part of the electric conducting unit is embedded in the biological sample holding unit”, for example.

However, the container disclosed in Patent Document 3 is such that a connecting unit being a part of the electric conducting unit is not covered and exposed. Therefore, for example, when a user measures the electric characteristic of the biological sample by using the container disclosed in Patent Document 3 and when a manufacturer manufactures the container disclosed in Patent Document 3, a risk of erroneous contact with the connecting unit is significantly high.

In a case where hands and fingers of the user, the manufacturer or the like erroneously make contact with the connecting unit, injury such as cut might occur and there is a problem from a viewpoint of safety of a product. Also, there might be a risk that the connecting unit is deformed or broken at the time of transfer or the like and a risk that the connecting unit breaks through a package of a product due to extra external force applied to the connecting unit. Furthermore, there is a problem that an unexpected substance such as dust in air adheres to the connecting unit and measuring accuracy at the time of electric characteristic measurement is deteriorated.

### CITATION LIST

### Patent Document

Patent Document 1: Japanese Patent Application Laid-Open No. 2009-042141
Patent Document 2: Japanese Patent Application Laid-Open No. 2010-181400
Patent Document 3: Japanese Patent Application Laid-Open No. 2014-115256

### SUMMARY OF THE INVENTION

### Problems to be Solved by the Invention

As described above, the connecting unit is exposed in the conventional electric measuring container of the biological sample, and it is difficult to decrease the risk of contact with the connecting unit.

Therefore, a principal object of the present technology is to provide the electric measuring cartridge capable of decreasing the risk of the contact with the connecting unit.

### Solutions to Problems

In order to solve the above-described problem, the inventors of the present application successfully decrease a risk of contact with a connecting unit to achieve the present technology by providing a protecting unit which protects the connecting unit on a cartridge as a result of serious study about a structure of the cartridge used when measuring an electric characteristic.

That is to say, in the present technology, first, an electric measuring cartridge at least provided with

a biological sample holding unit which accommodates a biological sample,

at least a pair of electrodes fixed to the biological sample holding unit,

a connecting unit which electrically connects the electrodes to an external circuit, and a protecting unit which protects the connecting unit is provided.

Also, the electric measuring cartridge according to the present technology may also be provided with a guiding unit fixed to the biological sample holding unit to guide insertion of the cartridge into a biological sample electric measuring device.

Furthermore, the guiding unit may also serve as the protecting unit.

In addition, in the electric measuring cartridge according to the present technology, a reagent may be enclosed in a part of the biological sample holding unit.

Also, although a material of the biological sample holding unit and/or the protecting unit is not especially limited, resin may be used.

Furthermore, although a type of the resin is not especially limited, one or more types of resin selected from polypropylene, polystyrene, acrylic, and polysulfone may be used.

In the present technology, the biological sample may be a blood sample.

Also, in a case where the biological sample is the blood sample, the electric measuring cartridge according to the present technology may be used for measuring a blood coagulated condition.

The present technology also provides a biological sample electric measuring device which measures an electric characteristic of a biological sample,

the biological sample electric measuring device at least provided with

a cartridge insertion portion into which an electric measuring cartridge at least provided with a biological sample holding unit which accommodates the biological sample, at least a pair of electrodes fixed to the biological sample holding unit, a connecting unit which electrically connects the electrodes to an external circuit, and a protecting unit which protects the connecting unit is inserted,

an applying unit which applies voltage to the connecting unit, and

a measuring unit which measures the electric characteristic of the biological sample.

Also, in the biological sample electric measuring device according to the present technology, in a case where the electric measuring cartridge is provided with a guiding unit fixed to the biological sample holding unit to guide insertion of the cartridge into the biological sample electric measuring device, the cartridge insertion portion may be provided with a guided unit with which the guiding unit is engaged.

Furthermore, the present technology also provides a biological sample electric measuring method for measuring an electric characteristic of a biological sample by using an electric measuring cartridge at least provided with

a biological sample holding unit which accommodates a biological sample,

at least a pair of electrodes fixed to the biological sample holding unit,

a connecting unit which electrically connects the electrodes to an external circuit, and

a protecting unit which protects the connecting unit.

### Effects of the Invention

An electric measuring cartridge according to the present technology is provided with a protecting unit which protects a connecting unit, so that a risk of contact with the connecting unit maybe decreased. Meanwhile, the effects are not necessarily limited to the effects herein described and may include any of the effects described in the present technology.

### BRIEF DESCRIPTION OF DRAWINGS

FIG. 1 is a schematic diagram schematically illustrating a first embodiment of an electric measuring cartridge 1 according to the present technology.

A of FIG. 2 is an end face schematic diagram as seen in an arrow a direction in FIG. 1, and B of FIG. 2 is an end face schematic diagram as seen in an arrow b direction in FIG. 1.

FIG. 3 is an end face schematic diagram schematically illustrating a second embodiment of the electric measuring cartridge 1 according to the present technology.

FIG. 4 is an end face schematic diagram schematically illustrating a third embodiment of the electric measuring cartridge 1 according to the present technology.

FIG. 5 is an end face schematic diagram schematically illustrating a fourth embodiment of the electric measuring cartridge 1 according to the present technology.

FIG. 6 is a schematic diagram schematically illustrating a first embodiment of a biological sample electric measuring device 10 according to the present technology.

FIG. 7 is a schematic diagram schematically illustrating a second embodiment of the biological sample electric measuring device 10 according to the present technology.

FIG. 8 is a schematic diagram schematically illustrating a first embodiment of a biological sample electric measuring kit K according to the present technology.

### MODE FOR CARRYING OUT THE INVENTION

Hereinafter, a preferred mode for carrying out the present technology is described with reference to the drawings. Meanwhile, the embodiment hereinafter described illustrates an example of a representative embodiment of the present technology, and the scope of the present technology is not narrowed by them. Meanwhile, the description is given in the following order.

### 1. Electric Measuring Cartridge 1

(1) Biological Sample Holding Unit 11

(2) Electrode 12

(3) Connecting Unit 13

(4) Protecting Unit 14

(5) Guiding Unit 15

(6) Sealing Unit 16

(7) Biological Sample S

(8) Reagent R

(9) Others
(a) Cutout 111
(b) Click 161

### 2. Biological Sample Electric Measuring Device 10

(1) Cartridge Insertion Portion 2

(2) Applying Unit 3

(3) Measuring Unit 4

(4) Guided Unit 5

(5) Analyzing Unit 6

(6) Others

### 3. Biological Sample Electric Measuring Kit K

(1) Biological Sample Introducing Member 7

### 4. Biological Sample Electric Measuring Method

### 1. Electric Measuring Cartridge 1

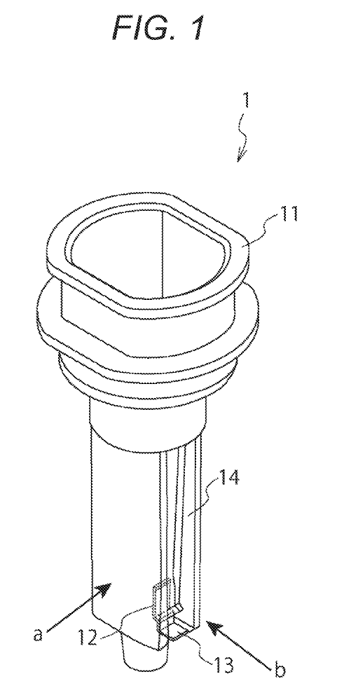

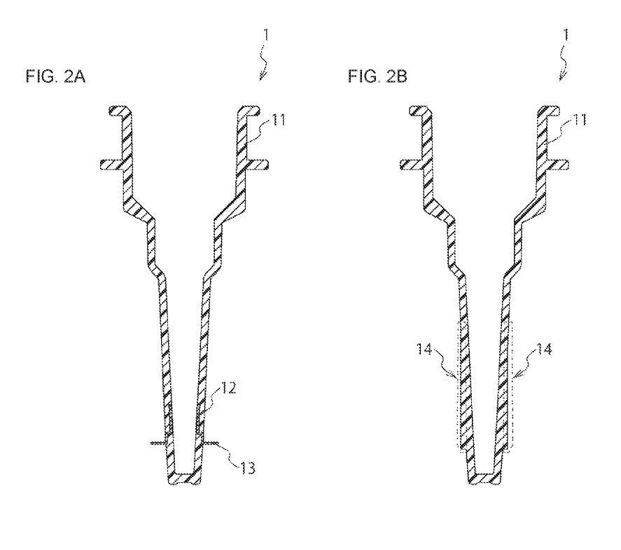

FIG. 1 is a schematic diagram schematically illustrating a first embodiment of an electric measuring cartridge 1 according to the present technology. A of FIG. 2 is an end face schematic diagram as seen in an arrow a direction in FIG. 1, and B of FIG. 2 is an end face schematic diagram as seen in an arrow b direction in FIG. 1.

The electric measuring cartridge 1 according to the present technology is a cartridge used for holding a biological sample S when measuring an electric characteristic of the biological sample S. The electric measuring cartridge 1 according to the present technology is at least provided with a biological sample holding unit 11, an electrode 12, a connecting unit 13, and a protecting unit 14 roughly. This may also be further provided with a guiding unit 15, a sealing unit 16 and the like as needed. Hereinafter, each portion is described in detail. Meanwhile, although the biological sample S is illustrated for the purpose of description in some drawings, the biological sample S is not always included in the electric measuring cartridge 1 according to the present technology.

(1) Biological Sample Holding Unit 11

The biological sample holding unit 11 is a portion in which the biological sample S to be measured is accommodated.

In the electric measuring cartridge 1 according to the present technology, a shape of the biological sample holding unit 11 is not especially limited; this may be freely designed according to a type of the biological sample S, a method of measuring the same, an electric measuring device to be used and the like. For example, there are a cylindrical body, a polygonal tubular body having a polygonal (triangular, square and so on) cross-shape, a conical body, a polygonal cone body having a polygonal (triangular, square and so on) cross-shape, a shape obtained by combining one or two or more of them, or the like.

In the present technology, it is preferable to select a shape in which at least a portion in which the electrode 12 to be described later is arranged is planar as the shape of the biological sample holding unit 11. A reason for this is hereinafter described in detail.

In general, the electrode used at the time of electric measurement often has a planar shape and a plate-like shape. In a case where a cylindrical shape is selected for the biological sample holding unit 11, the electrode 12 having the planar shape or the plate-like shape is attached to a curved portion, so that a manufacturing process becomes significantly complicated. Also, in a case where the electrode 12 having the planar shape or the plate-like shape is attached to the curved portion of the biological sample holding unit 11, it is highly possible that there is a step in a connecting portion between the biological sample holding unit 11 and the electrode 12 and measuring accuracy at the time of the electric measurement might be deteriorated. Therefore, it is possible to realize a simple manufacturing process of the electric measuring cartridge 1 and improvement in measuring accuracy by selecting the shape in which at least the portion in which the electrode 12 is arranged is planar in the biological sample holding unit 11.

In the present technology, various types of electric measurement are performed on the biological sample S in a state in which this is held in the biological sample holding unit 11. Therefore, the biological sample holding unit 11 is preferably configured to be sealable in the state in which the biological sample S is held therein. However, the sealable configuration is not necessarily required if it is possible to hold up time required for measuring various electric characteristics of the biological sample S and there is no effect on the measurement.

A specific method of introducing the biological sample S into the biological sample holding unit 11 or a specific method of sealing the same is not especially limited, and it is possible to introduce or seal by a free method according to the shape of the biological sample holding unit 11. For example, there is a method of introducing into the biological sample holding unit 11 by using a pipette and the like, a method of injecting the biological sample S with an injection needle penetrating an outer surface of the biological sample holding unit 11 and thereafter sealing by closing a portion through which the injection needle penetrates with grease and the like.

A material which may be used for the biological sample holding unit 11 according to the present technology is not especially limited; the biological sample holding unit 11 may be formed of resin in the present technology.

Also, a type of resin which may be used for the biological sample holding unit 11 is not especially limited; one or two or more types of resin applicable to holding of the biological sample S may be freely selected to be used. For example, there are hydrophobic and insulating polymer, co-polymer, and blended polymer such as polypropylene, polymethylmethacrylate, polystyrene, acrylic, polysulfone, and polytetrafluoroethylene.

In the present technology, it is preferable to form the biological sample holding unit 11 of one or more types of resin selected especially from polypropylene, polystyrene, acrylic, and polysulfone out of them. This is because the resins are characterized by low anticoagulant activity for blood, so that they maybe preferably used for measuring and the like in a case where a blood sample is selected as the biological sample S, for example.

(2) Electrode 12

The electrode 12 is a portion fixed to the biological sample holding unit 11 formed of at least a pair of electrodes. The electrode 12 makes contact with the biological sample S at the time of the electric measurement to apply required voltage to the biological sample S for electrically measuring a state of the biological sample S.

The electric measuring cartridge 1 according to the present technology may also be provided with one or more pairs of electrodes 12 according to the type of the biological sample S, the method of measuring the same, the electric measuring device to be used and the like. For example, in a case where a dielectric constant and impedance of the biological sample S are measured, it is possible to design one or more pairs of electrodes 12 for the biological sample holding unit 11. In this case, it is preferable to arrange the electrodes 12 so as to be parallel when measuring the electric characteristic of the biological sample S. However, it is also possible to arrange the electrodes 12 with an inclination of few degrees in consideration of a mold release property and the like in a case of performing insert molding and the like, for example.

The arrangement, shapes of the electrodes 12 and the like are not especially limited; it is possible to freely design according to the shape of the biological sample holding unit 11, the measuring method, the electric measuring device to be used and the like as long as the required voltage may be applied to the biological sample S. In the present technology, it is especially preferable that the electrode 12 is in contact with the biological sample S in a planar manner especially from a viewpoint of improving measuring efficiency. If there is a step on an inner wall of the biological sample holding unit 11, bubble and the like stays in the step portion, and reagent concentration becomes uneven in the step portion, so that this might adversely affect a measured value. It is possible to eliminate the adverse effect due to the bubble, uneven sample concentration, and the like by making a connecting portion between the biological sample holding unit 11 and the electrode 12 smooth, to improve the measuring accuracy at the time of the electric measurement.

A specific method by which the electrode 12 is brought into contact with the biological sample in a planar manner is not especially limited; for example, there is a method of forming the electrode 12 to be wide by which the electrode 12 is brought into contact with the biological sample S in a planar manner.

Although a method of fixing the electrode 12 to the biological sample holding unit 11 is not especially limited in the present technology, a method of integrally forming the biological sample holding unit 11 and the electrode 12 in a state in which a part of the electrode 12 is embedded in the biological sample holding unit 11 is preferable. A reason for this is hereinafter described in detail.

In a case where the electrode 12 is fixed to the biological sample holding unit 11 with an adhesive, for example, some types of adhesive to be used might adversely affect a characteristic of the biological sample S. For example, in a case where blood is selected as the biological sample S, some types of adhesive to be used might promote blood coagulation activity to adversely affect obj ective measurement. However, when the method of integrally forming the biological sample holding unit 11 and the electrode 12, that is to say, a method in which a fixing material such as the adhesive is not used for fixing the biological sample holding unit 11 and the electrode 12 is chosen, it is possible to eliminate the adverse effect on the biological sample S by the fixing material such as the adhesive and the measuring accuracy at the time of the electric measurement is improved.

Even in a case where using the fixing material with a little effect on the biological sample S, a bonding process by using the fixing material increases when manufacturing the cartridge for accommodating the biological sample S, so that productivity is deteriorated. However, if the method of integrally forming the biological sample holding unit 11 and the electrode 12 is chosen, it is not required to separately provide the bonding process in addition to a forming process of the biological sample holding unit 11. Therefore, the electric measuring cartridge 1 may be easily manufactured and the electric measuring cartridge 1 according to the present technology may be produced on a large scale at a low cost.

In the present technology, a specific method of integrally forming the biological sample holding unit 11 and the electrode 12 is not especially limited and a free method may be chosen. For example, in a case where the biological sample holding unit 11 is formed of resin, it is possible to integrally form the biological sample holding unit 11 and the electrode 12 by arranging the electrode 12 in a predetermined position when the resin is solidified from a melting state. More specifically, for example, there is a method of integrally forming the biological sample holding unit 11 and the electrode 12 by so-called insert molding in which the electrode 12 is inserted into a mold and the resin is poured around the same to make the electrode 12 and the resin integral.

Also, it is possible to simplify the manufacturing process of the electric measuring cartridge 1 by fixing the electrode 12 to the biological sample holding unit 11 at the same time when the biological sample holding unit 11 is formed. Therefore, it is possible to produce the electric measuring cartridge 1 according to the present technology on a large scale at a low cost. It is also possible to make the electric measuring cartridge 1 according to the present technology disposable, for example, by using this characteristic. By making the electric measuring cartridge 1 according to the present technology disposable, it becomes possible to save a step such as cleaning of the cartridge, so that the measurement may be performed with efficiency. Also, occurrence of a measuring error and the like due to another biological sample S remaining in the cartridge may be prevented and the measuring accuracy at the time of the electric measurement is improved.

Furthermore, although it is extremely rare, there is a case in where the biological sample S leaks out from a boundary between the biological sample holding unit 11 and the electrode 12 depending on a storage condition such as temperature and a measuring condition due to, for example, difference in distortion between the resin and an electric conductive material. Therefore, by providing a bent portion in a part of a structure of a portion fixed to the biological sample holding unit 11 on the electrode 12, it becomes possible to more certainly prevent leakage of the biological sample S from the boundary between the biological sample holding unit 11 and the electrode 12 as compared to a case without the bent portion. Also, fixing between the biological sample holding unit 11 and the electrode 12 becomes stronger and it is possible to form a solid electric measuring cartridge 1.

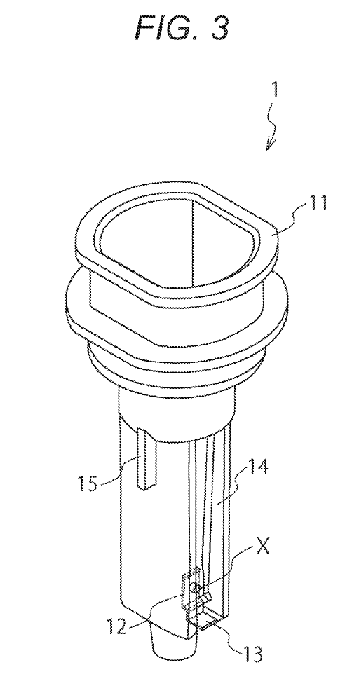

In addition, the electric measuring cartridge 1 according to the present technology may also have a configuration without resin on an outer portion of the cartridge of a pair of electrodes 12 as illustrated in a second embodiment in FIG. 3 (refer to X in FIG. 3). By such a structure, it is possible to correctly position the electrode 12 with respect to a desired position of the electric measuring cartridge 1 by positioning and fixing the electrode 12 with magnetic means such as a magnet when the biological sample holding unit 11 is formed, for example. Also, the electrode 12 is held when the biological sample holding unit 11 is formed and deformation of the electrode 12 may be prevented.

Meanwhile, not only the above-described magnetic means but also every means may be used as means of fixing the electrode 12 from outside the cartridge as long this is means capable of fixing the electrode 12 from outside the cartridge.

(3) Connecting Unit 13

The connecting unit 13 is a portion which electrically connects the electrode 12 to an external circuit. Therefore, in the present technology, apart of the connecting unit 13 has a structure connected to the electrode 12 as illustrated in the first embodiment in FIG. 1 and the like.

Arrangement, a shape of the connecting unit 13 and the like are not especially limited; they may be freely designed according to the shape of the biological sample holding unit 11, the measuring method, the electric measuring device to be used and the like as long as it is possible to electrically connect to the external circuit.

In the present technology, the electrode 12 and the connecting unit 13 are formed of an electric conductive material. A type of the electric conductive material used for the electrode 12 and the connecting unit 13 is not especially limited; one or two or more types of materials applicable to the measurement of the electric characteristic of the biological sample S may be freely selected to be used. For example, there is titanium, aluminum, stainless, platinum, gold, copper, graphite and the like.

In the present technology, it is preferable to form the electrode 12 and the connecting unit 13 especially of the electric conductive material including titanium out of them. This is because titanium is characterized by low anticoagulant activity for blood, so that this may be preferably used for measuring and the like in a case where the blood sample is selected as the biological sample S, for example.

(4) Protecting Unit 14

The protecting unit 14 is a portion which protects the connecting unit 13.

As described above, the connecting unit is exposed in the conventional electric measuring container of the biological sample, so that a risk that a user, a manufacturer and the like erroneously make contact with the connecting unit is very high. The electric measuring cartridge 1 according to the present technology is provided with the protecting unit 14 which protects the connecting unit 13, so that the risk that the user and the manufacturer make contact with the connecting unit 13 may be decreased. Therefore, erroneous contact of hands and fingers of the user, the manufacturer, and the like with the connecting unit 13 decreases and possibility of injury such as cut also decreases. Therefore, the electric measuring cartridge 1 according to the present technology is significantly useful from a viewpoint of safety of a product.

Also, a risk that the connecting unit 13 is deformed or broken at the time of transfer or the like and a risk that the connecting unit 13 breaks through a package of the product may be avoided by the protecting unit 14. Furthermore, a possibility that an unexpected substance such as dust in air adheres to the connecting unit 13 decreases by the protecting unit 14 and it is possible to improve the measuring accuracy at the time of the electric measurement.

The arrangement, the shape of the protecting unit 14 and the like are not especially limited and they may be freely designed according to the shape of the biological sample holding unit 11, the measuring method, the electric measuring device to be used and the like as long as the connecting unit 13 may be protected; however, the connecting unit 13 is the portion which electrically connects the electrode 12 to the external circuit as described above. Therefore, it is not possible to completely cover the connecting unit 13 with the protecting unit 14 in the present technology. Therefore, it is required to provide an opening on at least apart of the protecting unit 14 such that the connecting unit 13 is not prevented from making contact with the external circuit as illustrated in the first embodiment in FIG. 1 and the like, for example.

A material which may be used for the protecting unit 14 according to the present technology is not especially limited; in the present technology, it is possible to form the protecting unit 14 of resin.

Also, a type of the resin which may be used for the protecting unit 14 is not especially limited; one or two or more types of resin applicable to holding of the biological sample S may be freely selected to be used. For example, there are hydrophobic and insulating polymer, co-polymer, and blended polymer such as polypropylene, polymethylmethacrylate, polystyrene, acrylic, polysulfone, and polytetrafluoroethylene.

In the present technology, it is preferable to form the protecting unit 14 of one or more types of resin selected especially from polypropylene, polystyrene, acrylic, and polysulfone out of them. This is because the resins are characterized by low anticoagulant activity for blood, so that they may be preferably used for measuring and the like in a case where a blood sample is selected as the biological sample S, for example.

Meanwhile, although right and left protecting units 14 have the same size in the first embodiment illustrated in FIG. 1, it is also possible to make the sizes of the right and left protecting units 14 different. By making the sizes of the right and left protecting units 14 different, in a case where a bar code and the like is attached to the electric measuring cartridge 1 for management, for example, they may serve as a mark of a position to which the bar code and the like is attached, so that user convenience is improved.

(5) Guiding Unit 15

The electric measuring cartridge 1 according to the present technology may further be provided with the guiding unit 15 as illustrated in the second embodiment in FIG. 3. The guiding unit 15 is a portion which guides insertion of the electric measuring cartridge 1 into the biological sample electric measuring device.

By providing the guiding unit 15 on the electric measuring cartridge 1, the insertion into the biological sample electric measuring device becomes smooth. In a case where the bar code and the like is attached to the electric measuring cartridge 1 for management, for example, this may serve as the mark of the position to which the bar code and the like is attached. Therefore, the user convenience may be improved.

Also, in a case where a positioning mechanism to be described later is formed by using the guiding unit 15, the position of the electric measuring cartridge 1 in the biological sample electric measuring device maybe correctly determined and the measuring error due to displacement in position of the electric measuring cartridge 1 may be decreased.

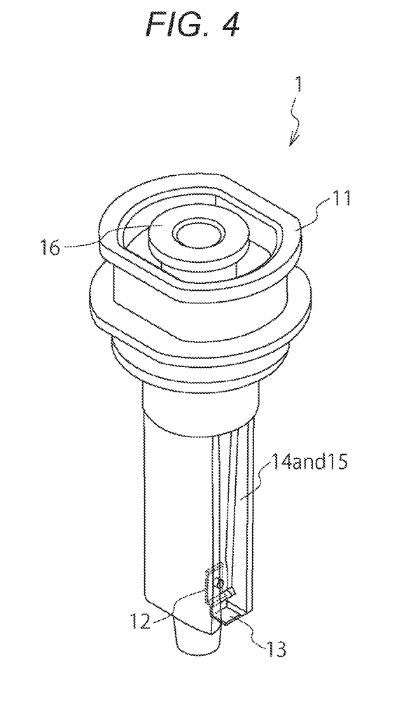

Also, in the present technology, the guiding unit 15 may also serve as the protecting unit 14 as illustrated in a third embodiment in FIG. 4. With this arrangement, the protecting unit 14 and the guiding unit 15 may be simultaneously formed, so that it is not required to separately provide a manufacturing process. Therefore, the electric measuring cartridge 1 may be easily manufactured and the electric measuring cartridge 1 according to the present technology maybe produced on a large scale at a low cost. Also, a configuration of the biological sample measuring device may also be simplified, so that this may contribute to realization of a compact device at a low cost.

The electric measuring cartridge 1 according to the present technology may be such that a reagent R is further enclosed in a part of the biological sample holding unit 11 as needed. In the present technology, a method of enclosing the reagent R is not especially limited; for example, there is a method of further providing the sealing unit 16 described hereinafter and the like, for example.

(6) Sealing Unit 16

FIG. 4 is a schematic diagram schematically illustrating the third embodiment of the electric measuring cartridge 1 according to the present technology further provided with the sealing unit 16. The sealing unit 16 is a portion which seals at least a part of the biological sample holding unit 11.

Since the electric measuring cartridge 1 according to the present technology is provided with the sealing unit 16, it is possible to enclose the reagent R in a sealed portion of the biological sample holding unit 11.

In a case where the electric measuring cartridge 1 according to the present technology is provided with the sealing unit 16, a shape of the sealing unit 16 is not especially limited as long as this may seal a part of the biological sample holding unit 11; this maybe freely designed depending on the type of the biological sample S, the method of measuring the same, the electric measuring device to be used and the like. For example, there are a cylindrical body, a polygonal tubular body having a polygonal (triangular, square and so on) cross-shape, a conical body, a polygonal cone body having a polygonal (triangular, square and so on) cross-shape, a shape obtained by combining one or two or more of them, or the like.

Also, the material which may be used for the sealing unit 16 is not especially limited; this may be formed of resin as the biological sample holding unit 11. Meanwhile, since the type of resin is similar to that described above, this is not herein described.

In a case where the electric measuring cartridge 1 according to the present technology is provided with the sealing unit 16, it is preferable to design such that the sealing unit 16 interrupts the sealed portion of the biological sample holding unit 11 and the electrode 12. This is because dust in air and the like which might deteriorate the measuring accuracy may be prevented from adhering to the electrode 12. Also, in a case where the electric measuring cartridge 1 is stored, carried or the like in a state in which the reagent R is enclosed in a part of the biological sample holding unit 11 in advance, it is possible to prevent the reagent R from being splattered to the inner wall of the biological sample holding unit 11 and the electrode 12. Therefore, it is possible to maintain an effective reagent amount for the biological sample S and decrease the measuring error and the like due to the reagent R remained in the electrode 12 at the time of the electric characteristic measurement.

Meanwhile, in a case where the electric measuring cartridge 1 according to the present technology is provided with the sealing unit 16, the electric characteristic measuring cartridge 1 is in a state in which the biological sample S is held in the biological sample holding unit 11 and the sealing unit 16 is removed at the time of the electric characteristic measurement.

(7) Biological Sample S

The biological sample S which may be measured by the present technology is not especially limited and may be freely selected. For example, there is a blood sample containing a blood component such as whole blood, blood plasma, or dilution thereof and/or a drug-added substance and the like. Also, in a case where the blood sample is selected as the biological sample S, it is also possible to use the electric measuring cartridge 1 according to the present technology for measuring a blood coagulated condition.

(8) Reagent R

In a case where the electric measuring cartridge 1 according to the present technology is such that the reagent R may be enclosed in a part of the biological sample holding unit 11, the reagent R which may be enclosed in a part of the biological sample holding unit 11 is not especially limited and may be freely selected. For example, there is the reagent in a gas state, a solid state, a liquid state and the like. More specifically, in a case where the blood sample is selected as the biological sample S, there are an anticoagulant, a coagulation starting agent and the like.

In a case where the electric measuring cartridge 1 according to the present technology is such that the reagent R may be enclosed in a part of the biological sample holding unit 11, the electric measuring cartridge 1 maybe carried, stored and the like in the state in which the reagent R is enclosed in advance. In this case, the measurement may be immediately started only with a process of opening the electric measuring cartridge 1 immediately before performing the measurement and introducing the biological sample S to be measured into the biological sample holding unit 11. Therefore, dust in air and the like which might deteriorate the measuring accuracy may be prevented from mixing into the biological sample holding unit 11 and the measuring accuracy is improved. Furthermore, an operation process until the measurement is started is decreased and the user convenience is improved.

It is also possible to temporarily store the electric measuring cartridge 1 by a method of cooling, freezing, freeze-drying and the like in the state in which the reagent R is enclosed therein depending on a type of the reagent used as the reagent R.

(9) Others

The electric measuring cartridge 1 according to the present technology may also be provided with a fixing mechanism in a case where the electric measuring cartridge 1 is provided with the sealing unit 16. In the present technology, the fixing mechanism is a mechanism which fixes the biological sample holding unit 11 to the sealing unit 16 when the sealing unit 16 seals at least a part of the biological sample holding unit 11. When the electric measuring cartridge 1 is provided with the fixing mechanism, the sealing unit 16 is prevented from escaping from the biological sample holding unit 11 and it is possible to stably maintain a sealing state of a part of the biological sample holding unit 11 and the sealing unit 16.

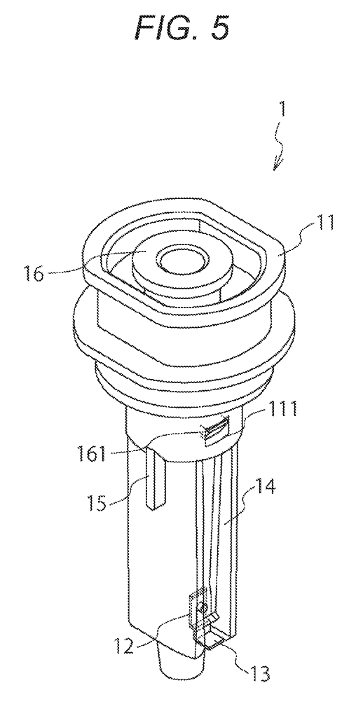

A specific configuration of the fixing mechanism is not especially limited; there is a case in which this is formed of a cutout 111 provided on the biological sample holding unit 11 and a click 161 fitting in the cutout 111 provided on the sealing unit 16 as illustrated in a fourth embodiment in FIG. 5, for example. The guiding unit 15 may also serve as the cutout 111.

It is also possible to design the fixing mechanism so as not to be able to fix again after the fixing is released. If this cannot fix again after the fixing is released, in the electric measuring cartridge 1 once opened, the sealing unit 16 cannot fit in the biological sample holding unit 11 again. Therefore, it becomes possible to check an opening history of the electric measuring cartridge 1. Therefore, in a case where the electric measuring cartridge 1 is distributed in the state in which the reagent R is enclosed therein, for example, a quality thereof may be secured.

(a) Cutout 111

The cutout 111 is a portion provided on the biological sample holding unit 11 in which the click 161 fits. A shape of the cutout 111 is not especially limited; there is a hole, a groove and the like provided on the biological sample holding unit 11, for example. More specifically, there is a hole provided so as to penetrate from the inner wall to an outer wall of the biological sample holding unit 11, for example.

(b) Click 161

The click 161 is a portion provided on the sealing unit 16 to fit in the cutout 111. A shape of the click 161 is not especially limited; there is a projection provided on the sealing unit 16, for example.

The click 161 may also be designed to have flexibility. Specifically, there is a method of forming the click 161 of a material having flexibility such as resin, for example. When the click 161 has flexibility, this easily fits in the cutout 111 and the user convenience is improved.

Furthermore, the click 161 may be designed to be deformed or cut such that this cannot fix again when the fixing of the fixing mechanism is released. A mechanism to deform or cut the click 161 is not especially limited; there is a method of melting and the like the click 161 by chemical means using heat and the like to deform the same and a method of cutting the click 161 by physical means, for example. When the click 161 is deformed or cut, it becomes impossible that the click 161 fits in the cutout 111 again. Therefore, it also becomes impossible that the sealing unit 16 fits in the biological sample holding unit 11 again and the quality of the electric measuring cartridge 1 may be secured.

In the present technology, resin may be used also for the cutout 111 and the click 161 as in the biological sample holding unit 11 and the sealing unit 16. Meanwhile, since the type of resin is similar to that described above, this is not herein described.

Meanwhile, although the electrode 12, the cutout 111, and the click 161 are located on the same side surface of the biological sample holding unit 11 in the fourth embodiment illustrated in FIG. 5, it is also possible that the electrode 12, the cutout 111, and the click 161 are located on different side surfaces of the biological sample holding unit 11. By arranging the electrode 12, the cutout 111, and the click 161 on the different side surfaces of the biological sample holding unit 11, it becomes possible to avoid the electrode 12 from erroneously making contact with a sealing releasing mechanism when the electric measuring cartridge 1 is inserted into a cartridge insertion portion 2 of an electric measuring device 10 to be described later.

### 2. Biological Sample Electric Measuring Device 10

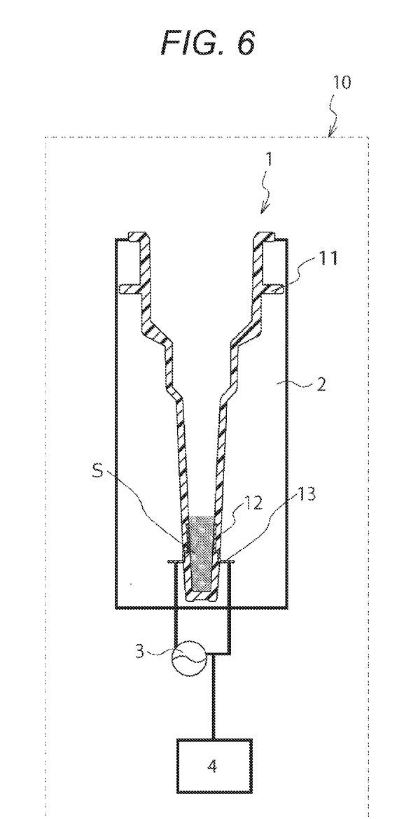

FIG. 6 is a schematic diagram schematically illustrating the first embodiment of the biological sample electric measuring device 10 according to the present technology. In this embodiment, the electric measuring cartridge 1 (refer to A of FIG. 2) according to the above-described first embodiment is used.

The biological sample electric measuring device 10 according to the present technology is at least provided with the electric measuring cartridge 1 described above, the cartridge insertion portion 2, an applying unit 3, and a measuring unit 4 roughly. It is also possible to be further provided with a guided unit 5, an analyzing unit 6 and the like as needed. Hereinafter, each portion is described in detail. Meanwhile, since the electric measuring cartridge 1 is similar to that described above, this is not herein described.

(1) Cartridge Insertion Portion 2

The cartridge insertion portion 2 is a portion into which the electric measuring cartridge 1 according to the present technology is inserted. The cartridge insertion portion 2 maybe freely designed according to the shape and the like of the electric measuring cartridge 1.

The cartridge insertion portion 2 may also be provided with a temperature adjusting mechanism. In the present technology, the temperature adjusting mechanism is a mechanism which keeps constant temperature of the biological sample S held in the biological sample holding unit 11. Since the biological sample electric measuring device 10 is provided with the temperature adjusting mechanism, the temperature of the biological sample S becomes constant and it is possible to decrease the measuring error due to temperature change of the biological sample S.

A specific configuration of the temperature adjusting mechanism is not especially limited; for example, the cartridge insertion portion 2 is formed of a material which may keep heat.

(2) Applying Unit 3

The applying unit 3 is a portion which applies voltage to the connecting unit 13 of the electric measuring cartridge 1 according to the present technology. Specifically, the applying unit 3 applies the voltage to the connecting unit 13 of the electric measuring cartridge 1 while making a time point at which this receives an order to start measuring or a time point at which the biological sample electric measuring device 10 is powered on a starting point. In this case, the applying unit 3 applies alternating voltage of a predetermined frequency to the connecting unit 13 at a set measuring interval. Meanwhile, the voltage applied by the applying unit 3 may also be direct voltage according to the electric characteristic to be measured.

(3) Measuring Unit 4

The measuring unit 4 is a portion which measures the electric characteristic of the biological sample S. Specifically, this measures the electric characteristic such as a complex dielectric constant (hereinafter, also referred to as “dielectric constant”) and frequency dispersion thereof while making the time point at which this receives the order to start measuring or the time point at which the biological sample electric measuring device 10 is powered on. For example, in a case where the dielectric constant is measured, the measuring unit 4 measures current or impedance between the electrodes 12 of the electric measuring cartridge 1 with a predetermined period to derive the dielectric constant from the measured value. It is possible to use known function and relational expression indicating a relationship between the current or impedance and the dielectric constant for deriving the dielectric constant.

(4) Guided Unit 5

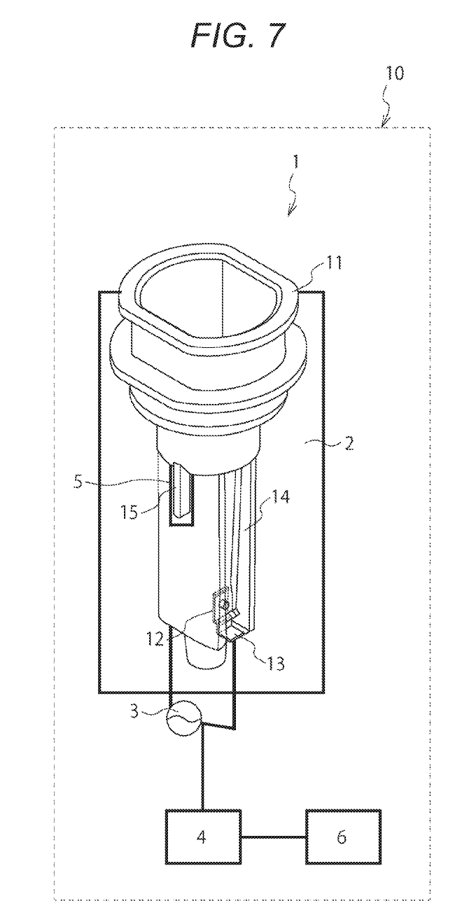

In a case where the electric measuring cartridge 1 according to the present technology is provided with the guiding unit 15 as illustrated in the second embodiment in FIG. 3, the biological sample electric measuring device 10 may further be provided with the guided unit 5 as illustrated in the second embodiment in FIG. 7. The guided unit 5 is a portion with which the guiding unit 15 is engaged.

Since the biological sample electric measuring device 10 is provided with the guided unit 5, the electric measuring cartridge 1 may be smoothly inserted into the biological sample electric measuring device 10 and the user convenience is improved. Also, in a case where the positioning mechanism to be described later is formed by using the guiding unit 15 and the guided unit 5, the position of the electric measuring cartridge 1 in the biological sample electric measuring device 10 may be correctly determined and the measuring error due to the displacement in position of the electric measuring cartridge 1 may be decreased.

(5) Analyzing Unit 6

The biological sample electric measuring device 10 according to the present technology may further be provided with the analyzing unit 6 as illustrated in the second embodiment in FIG. 7. The analyzing unit 6 is a portion which receives electric characteristic data of the biological sample S derived from the measuring unit 4 to, for example, determine a physical property of the biological sample S.

Specifically, the electric characteristic data of the biological sample S derived from the measuring unit 4 is given to the analyzing unit 6 at a measuring interval and the analyzing unit 6 receives the electric characteristic data given from the measuring unit 4 to start determining the physical property of the biological sample S. Also, the analyzing unit 6 gives notice of a result of, for example, the physical property determination and/or dielectric constant data of the biological sample S. This notice may be made by graphical display on a monitor or printing on a predetermined medium, for example.

(6) Others

The biological sample electric measuring device 10 according to the present technology may also be provided with the positioning mechanism. In the present technology, the positioning mechanism is a mechanism which determines the position of the electric measuring cartridge 1. When the biological sample electric measuring device 10 is provided with the positioning mechanism, the position of the electric measuring cartridge 1 in the biological sample electric measuring device 10 may be correctly determined and a contacting position of the connecting unit 13 and the applying unit 3 becomes correct, so that the measuring error due to the displacement in position of the electric measuring cartridge 1 may be decreased.

A specific configuration of the positioning mechanism is not especially limited; for example, a positioning pin which positions the electric measuring cartridge 1 with respect to the biological sample electric measuring device 10 in a height direction is installed, for example. It is also possible to form the positioning mechanism by using the guiding unit 15 and the guided unit 5.

The biological sample electric measuring device 10 according to the present technology may also be provided with the sealing releasing mechanism in a case where the electric measuring cartridge 1 is such that the reagent R may be enclosed in a part of the biological sample holding unit 11. In the present technology, the sealing releasing mechanism is a mechanism which releases the sealing state of at least a part of the biological sample holding unit 11. Since the biological sample electric measuring device 10 is provided with the sealing releasing mechanism, a risk that unnecessary shock is given to the electric measuring cartridge 1 and that extra external force is applied to the biological sample holding unit 11 or the sealing unit 16 decreases. Also, in a case where the sealing state of at least a part of the biological sample holding unit 11 may be smoothly released by the sealing releasing mechanism, when the reagent R is enclosed in a part of the biological sample holding unit 11 in advance, the reagent R is prevented from splattering to a cartridge wall surface and the like, so that the user convenience and measuring accuracy are improved.

A specific configuration of the sealing releasing mechanism is not especially limited, and in a case where the electric measuring cartridge 1 is provided with the fixing mechanism (for example, cutout 111 and click 161), for example, the fixing of the biological sample holding unit 11 and the sealing unit 16 by the fixing mechanism is released by using chemical means using heat and the like, physical means, and the like. More specifically, there is a method of installing a releasing pin on a side of the biological sample electric measuring device 10 when the electric measuring cartridge 1 is inserted into the cartridge insertion portion 2, for example.

### 3. Biological Sample Electric Measuring Kit K

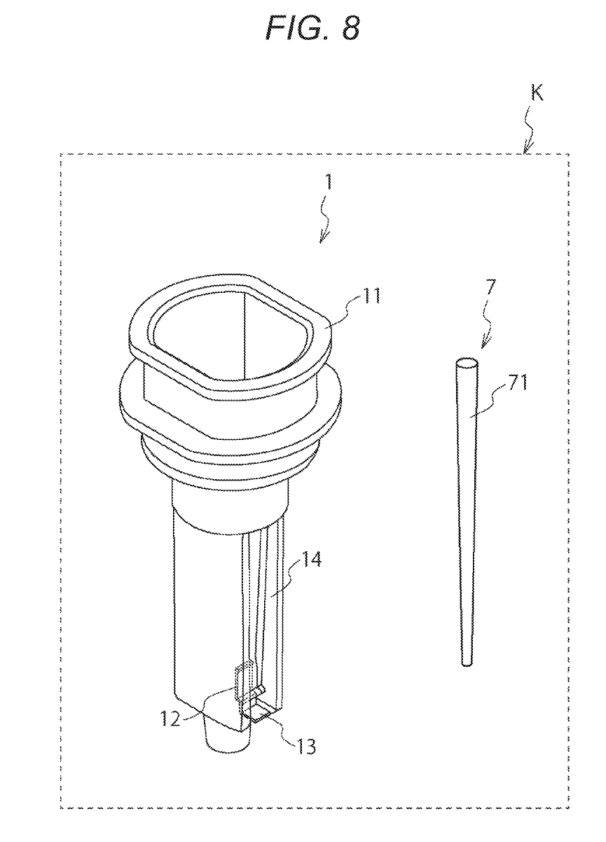

FIG. 8 is a schematic diagram schematically illustrating a first embodiment of an electric measuring kit K according to the present technology. In this embodiment, the electric measuring cartridge 1 according to the first embodiment described above is used.

The electric measuring kit K according to the present technology is at least provided with the above-described electric measuring cartridge 1 and a biological sample introducing member 7 roughly. This is hereinafter described in detail. Meanwhile, since the electric measuring cartridge 1 is similar to that described above, this is not herein described.

(1) Biological Sample Introducing Member 7

The biological sample introducing member 7 is a member for introducing the biological sample S into the biological sample holding unit 11. For example, as illustrated in the first embodiment in FIG. 8, there is a pipette-like chip 71 and the like. More specifically, a suction mechanism (pipetter and the like) is provided on the biological sample electric measuring device 10 and the pipette-like chip 71 may be attached to the suction mechanism to introduce the biological sample S.

The biological sample introducing member 7 according to the present technology is not limited to the pipette-like chip 71 illustrated in FIG. 8; this may be freely selected according to the type of the biological sample S, the method of measuring the same, the electric measuring device and the like as long as this is an entire or a part of a tool which may introduce the biological sample S into the biological sample holding unit 11. For example, there is an injection needle and the like in addition to the pipette-like chip 71.

The biological sample introducing member 7 may also be made disposable as the electric measuring cartridge 1. By making the biological sample introducing member 7 disposable, it is possible to save a step such as cleaning of the tool used for introducing the biological sample S and the user convenience may be improved and the measurement may be performed with efficiency. Also, occurrence of the measuring error and the like due to another biological sample S remaining in the tool used for introducing the biological sample S and the measuring accuracy at the time of the electric measurement is improved.

### 4. Biological Sample Electric Measuring Method

The electric measuring cartridge 1 according to the present technology may be preferably used for a biological sample electric measuring method for measuring the electric characteristic of the biological sample S. In the biological sample electric measuring method according to the present technology, the electric characteristic which may be measured is not especially limited; this maybe freely measured according to the type of the biological sample S, the physical property to be analyzed and the like. For example, the dielectric constant, impedance and the like may be measured.

By using the biological sample electric measuring method according to the present technology, it is possible to analyze the blood coagulated condition and a sedimentation condition from the measured value of the dielectric constant and impedance in a case where blood is selected as the biological sample S, for example. More specifically, it is possible to extract a parameter indicating respective characteristic from a plurality of measured values of the dielectric constant and/or impedance received within an analyzing period and analyze the blood coagulated condition and the sedimentation condition on the basis of comparison between the parameter and a reference value which determines a reference of an elevation in blood coagulation function and sedimentation process progression. Therefore, the measuring accuracy at the time of the electric measurement is improved.

Meanwhile, the present technology may also have a following configuration.

(1) An electric measuring cartridge at least including:

a biological sample holding unit which accommodates a biological sample;

at least a pair of electrodes fixed to the biological sample holding unit;

a connecting unit which electrically connects the electrodes to an external circuit; and a protecting unit which protects the connecting unit.

(2) The electric measuring cartridge according to (1), further including: a guiding unit fixed to the biological sample holding unit to guide insertion of the cartridge into a biological sample electric measuring device.

(3) The electric measuring cartridge according to (2), in which the guiding unit also serves as the protecting unit.

(4) The electric measuring cartridge according to any one of (1) to (3), in which a reagent is enclosed in a part of the biological sample holding unit.

(5) The electric measuring cartridge according to any one of (1) to (4), in which the biological sample holding unit and/or the protecting unit is formed of resin.

(6) The electric measuring cartridge according to (5), in which the resin is one or more types of resin selected from polypropylene, polystyrene, acrylic, and polysulfone.

(7) The electric measuring cartridge according to any one of (1) to (6), in which the biological sample is a blood sample.

(8) The electric measuring cartridge according to (7) used for measuring a blood coagulated condition.

(9) A biological sample electric measuring device which measures an electric characteristic of a biological sample,

the biological sample electric measuring device at least including:

a cartridge insertion portion into which an electric measuring cartridge at least provided with a biological sample holding unit which accommodates the biological sample, at least a pair of electrodes fixed to the biological sample holding unit, a connecting unit which electrically connects the electrodes to an external circuit, and a protecting unit which protects the connecting unit is inserted;

an applying unit which applies voltage to the connecting unit; and

a measuring unit which measures the electric characteristic of the biological sample.

(10) The biological sample electric measuring device according to (9), in which

the electric measuring cartridge is provided with a guiding unit fixed to the biological sample holding unit to guide insertion of the cartridge into the biological sample electric measuring device, and

the cartridge insertion portion is provided with a guided unit with which the guiding unit is engaged.

(11) A biological sample electric measuring method which measures an electric characteristic of a biological sample by using an electric measuring cartridge at least provided with

a biological sample holding unit which accommodates the biological sample,

at least a pair of electrodes fixed to the biological sample holding unit,

a connecting unit which electrically connects the electrodes to an external circuit, and

a protecting unit which protects the connecting unit.

### REFERENCE SIGNS LIST

1 Electric measuring cartridge
11 Biological sample holding unit
111 Cutout
12 Electrode
13 Connecting unit
14 Protecting unit
15 Guiding unit
16 Sealing unit
161 Click
10 Biological sample electric measuring device
2 Cartridge insertion portion
3 Applying unit
4 Measuring unit
5 Guided unit
6 Analyzing unit
S Biological sample
K Biological sample electric measuring kit
7 Biological sample introducing member
71 Pipette-like chip

1. An electric measuring cartridge at least comprising:
a biological sample holding unit which accommodates a biological sample;
at least a pair of electrodes fixed to the biological sample holding unit;
a connecting unit which electrically connects the electrodes to an external circuit; and a protecting unit which protects the connecting unit.

2. The electric measuring cartridge according to claim 1, further comprising: a guiding unit fixed to the biological sample holding unit to guide insertion of the cartridge into a biological sample electric measuring device.

3. The electric measuring cartridge according to claim 2, wherein the guiding unit also serves as the protecting unit.

4. The electric measuring cartridge according to claim 1, wherein a reagent is enclosed in a part of the biological sample holding unit.

5. The electric measuring cartridge according to claim 1, wherein the biological sample holding unit and/or the protecting unit is formed of resin.

6. The electric measuring cartridge according to claim 5, wherein the resin is one or more types of resin selected from polypropylene, polystyrene, acrylic, and polysulfone.

7. The electric measuring cartridge according to claim 1, wherein the biological sample is a blood sample.

8. The electric measuring cartridge according to claim 7, wherein the electric measuring cartridge is used for measuring a blood coagulated condition.

9. A biological sample electric measuring device which measures an electric characteristic of a biological sample,
the biological sample electric measuring device at least comprising:
a cartridge insertion portion into which an electric measuring cartridge at least provided with a biological sample holding unit which accommodates the biological sample, at least a pair of electrodes fixed to the biological sample holding unit, a connecting unit which electrically connects the electrodes to an external circuit, and a protecting unit which protects the connecting unit is inserted;
an applying unit which applies voltage to the connecting unit; and
a measuring unit which measures the electric characteristic of the biological sample.

10. The biological sample electric measuring device according to claim 9, wherein
the electric measuring cartridge is provided with a guiding unit fixed to the biological sample holding unit to guide insertion of the cartridge into the biological sample electric measuring device, and
the cartridge insertion portion is provided with a guided unit with which the guiding unit is engaged.

11. A biological sample electric measuring method configured to measure an electric characteristic of a biological sample by using an electric measuring cartridge at least provided with
a biological sample holding unit which accommodates the biological sample,
at least a pair of electrodes fixed to the biological sample holding unit,
a connecting unit which electrically connects the electrodes to an external circuit, and
a protecting unit which protects the connecting unit.
